(19) United States
(12) Patent Application Publication
(10) Pub. No.: US 2005/0004377 A1
(43) Pub. Date: Jan. 6, 2005

(54) Methods for producing amino substituted chromanes and intermediates therefor

(76) Inventors: Robert Scarborough, Half Moon Bay, CA (US); Panos Kalaritis, Florence, SC (US); George Yiannikouros, Florence, SC (US); Michel P. Cruskie JR., Florence, SC (US); Dezhi Sha, Florence, SC (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/276,838

(22) PCT Filed: May 16, 2001
(86) PCT No.: PCT/US01/15695

Related U.S. Application Data
(60) Provisional application No. 60/204,835, filed on May 17, 2000.
(60) Provisional application No. 60/208,765, filed on Jun. 2, 2000.

(51) Int. Cl. C07D 311/74

(52) U.S. Cl. 549/404

(57) ABSTRACT

Disclosed are process steps and processes for producing chromane compounds, preferably 2-(6-amino-chroman-2yl) acetic acid esters which are intermediates for producing platelet aggregation inhibitors and/or are themselves potent platelet aggregation inhibitors.

### FIELD OF THE INVENTION

This invention relates to processes for producing chromane compounds, preferably amino substituted 2-(chroman-2-yl) acetic acid esters which are intermediates for producing platelet aggregation inhibitors and/or are themselves potent platelet aggregation inhibitors.

### BACKGROUND OF THE INVENTION

One process for making benzopyrans or chromanes from coumarin derivatives is described in U.S. Pat. No. 5,731,324 at columns 101-103. However, that process involves chromatography as a purification step, which does not scale well commercially. The unprotected amino derivative bicyclic compound is found on on column 147.

Therefore, there is a need for improved processes for producing compounds that are useful as intermediates in processes for producing platelet aggregation inhibitors. There is a particular need for improved processes for making compounds having the benzo ring of the benzopyrans substituted by an amino group or a protected amino group. Such intermediates are useful for coupling with a carbonyl group to produce a carboxamide link and result in compounds that are useful platelet aggregation inhibitors or in intermediates for forming platelet aggregation inhibitors. Also needed is a process to produce relatively inexpensively large quantities of chromone intermediates that are useful for being resolved by conventional processes to produce benzopyran or 2-chromane derivatives wherein the chiral center at the two position of the saturated pyran ring portion of the bicyclic ring structure can be resolved into racemic mixtures (R/S) that are enriched with one of the R or S enantiomers or to produce substantially pure compositions of a single enantiomer (R or S enantiomer). Due to inherent losses of up to 50% or more of the starting materials (assuming a 50/50 R/S racemate) during enantiomeric resolution, there is a need for a process which is efficient enough to be scaled to an industrial level for inexpensively producing large quantities of a desired intermediate compound or large quantities of final 2-(chroman-2-yl) acetic acid ester compounds that are useful in the anticoagulant field.

Accordingly, there continues to be a need for a process that is adaptable to commercially scaleable production of such chromanes.

### SUMMARY OF THE INVENTION

The present invention relates to processes for producing chromane compounds, preferably amino substituted 2-(chroman-2-yl) acetic acid esters which are intermediates for producing therapeutic agents, or are themselves therapeutic agents, for disease states in mammals that have disorders caused by or impacted by platelet dependent narrowing of the blood supply.

In accordance with a preferred embodiment, there is provided a process for making a compound according to the formula: 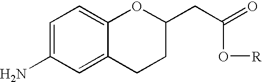
wherein R is H or an alkyl group. The method comprises:
(a) reacting 2-chloro-5-nitrobenzoic acid with a compound capable of halogenating the acid to form 2-chloro-5-nitrobenzoic acid halide as follows: 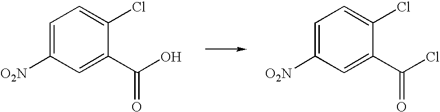
   wherein the halogenating agent is a member selected from the group consisting of a metallic acid halide, thionyl halide, or an organic halide donor compound;
(b) coupling 2-chloro-5-nitrobenzoyl chloride of the product from (a) above with acetonide (2,2,6-trimethyl-1,3-dioxin-4-one) to produce a ketone, in a lithium salt base in an acceptable organic solvent to produce 6-[2-(2-chloro-5-nitrophenyl)-2-oxoethyl]-2,2-dimethyl-1,3-dioxine-4-one, as follows: 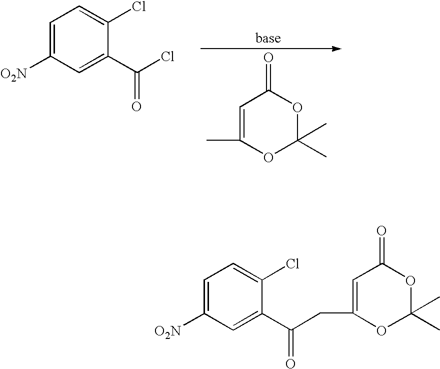
(c) opening the 1,3-dioxine ring of the product from (b) above at the 1 position and condensing the with the halogen atom to effect ring closure by heating the the product from (b) above to about 80° C. in tert butyl alcohol under nitrogen atmosphere to obtain t-butyl (6-nitro-4-oxo-2-chromen-2-yl)acetate (2), as follows: 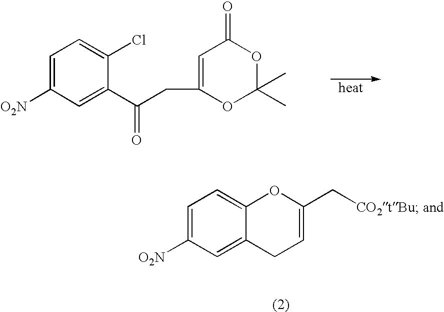
(d) reducing the 4-oxo group, 6-nitro group and 2-3 alkene bond of the chromenone ring of the product from (c) above in a single step or in separate steps as follows:

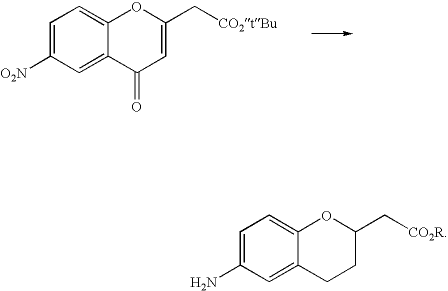

In a preferred embodiment, (d) comprises (d1) and (d2) wherein
(d1) reducing at least the 6-nitro group in the presence of glacial acetic acid followed by hydrogenation using 10% palladium on carbon in the presence of trifluoro acetic acid to from the 6-acetamido group as follows: 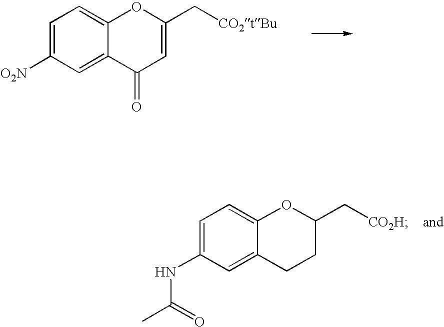
(d2) removing the protecting group from the 6-amino group by acidification and forming the final product as the free acid or ester as follows:

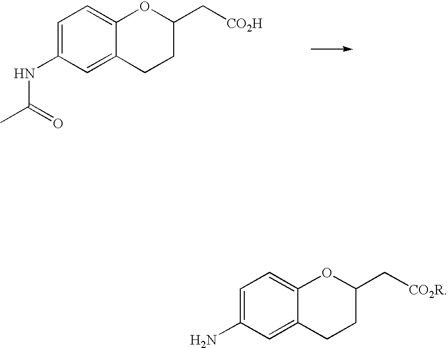

The resulting ester may be converted to its corresponding acid or to another ester by methods known to those skilled in the art. Salts of the acid or ester compounds, including acid halide salts, may also be prepared.

### DETAILED DESCRIPTION OF THE PREFERRED EMBODIMENTS

As mentioned above, preferred compounds produced using the methods disclosed herein have utility as intermediates for producing therapeutic agents or as therapeutic agents for disease states in mammals which have disorders that are due to platelet dependent narrowing of the blood supply, such as atherosclerosis and arteriosclerosis, acute myocardial infarction, chronic stable angina, unstable angina, transient ischemic attacks and strokes, peripheral vascular disease, arterial thrombosis, preclampsia, embolism, restenosis following angioplasty, carotid endarterectomy, anastomosis of vascular grafts, etc. These conditions represent a variety of disorders thought to be initiated by platelet activation on vessel walls.

Platelet adhesion and aggregation is believed to be an important part of thrombus formation. This activity is mediated by a number of platelet adhesive glycoproteins. The binding sites for fibrinogen, fibronectin and other clotting factors have been located on the platelet membrane glycoprotein complex IIb/IIIa. When a platelet is activated by an agonist such as thrombin the GPIIb/IIIa binding site becomes available to fibrinogen, eventually resulting in platelet aggregation and clot formation. Thus, intermediate compounds for producing compounds that effective in the inhibition of platelet aggregation and reduction of the incidence of clot formation are useful intermediate compounds.

The compounds produced according to the methods disclosed herein may also be used as intermediates to form compounds that may be administered in combination or concert with other therapeutic or diagnostic agents. In certain preferred embodiments, the compounds produced by the intermediates according to the present invention may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds produced from the intermediates according to the present invention may act in a synergistic fashion to prevent reocclusion following a successful thrombolytic therapy and/or reduce the time to reperfusion. Such compounds may also allow for reduced doses of the thrombolytic agents to be used and therefore minimize potential hemorrhagic side-effects. Such compounds can be utilized in vivo, ordinarily in mammals such as primates, (e.g. humans), sheep, horses, cattle, pigs, dogs, cats, rats and mice, or in vitro.

The starting materials and other reagents used in the processes disclosed are commercially available from chemical vendors such as Aldrich, Lancaster, TCI, Bachem Biosciences, and the like, or may be readily synthesized by known procedures, for example, those present in the chemical literature.

Reactions are carried out in standard laboratory glassware and reaction vessels under reaction conditions of standard temperature and pressure, except where otherwise indicated, or is well-known in literature available in the art. Further, the above procedures of the claimed invention processes my be carried out on a commercial scale by utilizing reactors and standard scale-up equipment available in the art for producing large amounts of compounds in the commercial environment. Such equipment and scale-up procedures are well-known to the ordinary practitioner in the field of commercial chemical production.

During the synthesis of these compounds, amino or acid functional groups may be protected by blocking groups to prevent undesired reactions with these groups during certain procedures. Use of other blocking groups or protecting groups known in the art, but not described specifically herein are also contemplated. The application and removal of such blocking groups by procedures such as acidification or hydrogenation are known in the art.

### Preferred Process for Preparing Ethyl 2-(6-amino-chroman-2-yl)acetate

In accordance with a preferred embodiment, there is provided a process that utilizes a 5-chloro-5-nitrobenzoic acid to produce ethyl 2-(6-amino-chroman-2-yl)acetate as set forth in reaction Scheme I below.

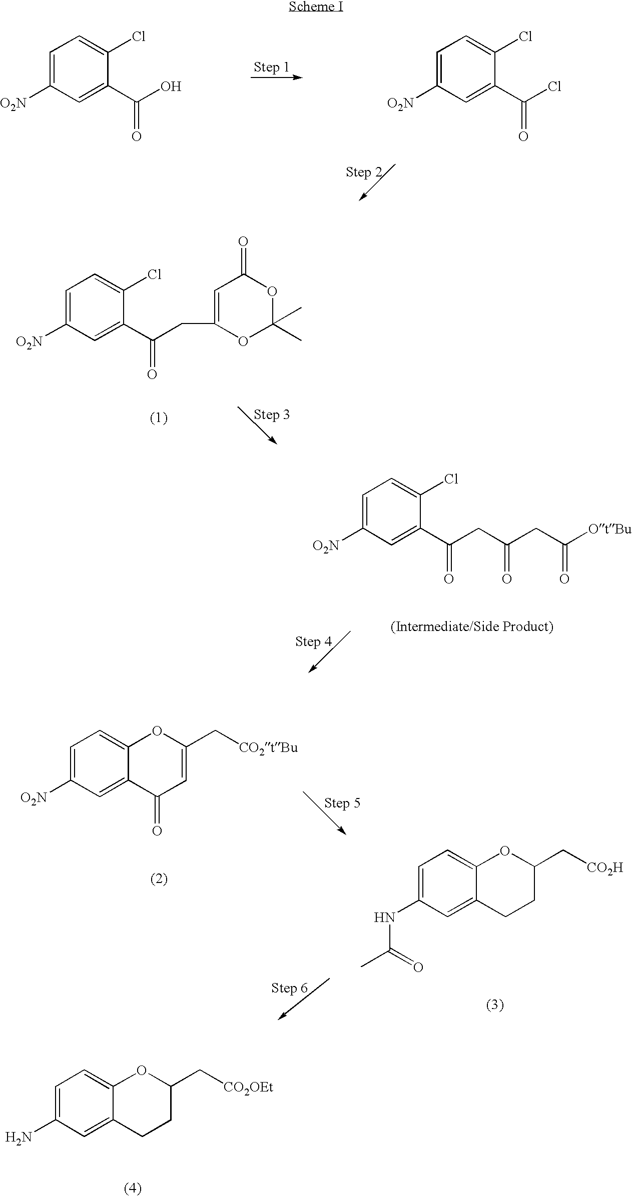

While an ethyl group was used to form the ester of the acetic acid side chain in the last step, the ethyl group can be replaced by H or another group capable of forming an ester selected from lower alkyl, lower alkenyl, lower alkynyl, phenyl, cinnamyl or other ester groups.

In either event, the protected amine benzopyran compound or the free amine benzopyran compound can be coupled to a cyanobenzoyl chloride group as described on pages 147 and 148 of U.S. Pat. No. 5,731,324, for example. The ester group of the acetic acid side chain can be optionally changed, before of after the coupling step.

Further, the above process can be modified to produce a formyl, propyl or butyl side chain or the like, by esterifying with a different alcohol starting material.

Referring back to Scheme I, the individual reaction steps illustrated therein will now be discussed in more detail. In the first step 2-chloro-5-nitrobenzoic acid is converted to the 2-chloro-5-nitrobenzoyl halide, for example, with thionyl chloride as follows:

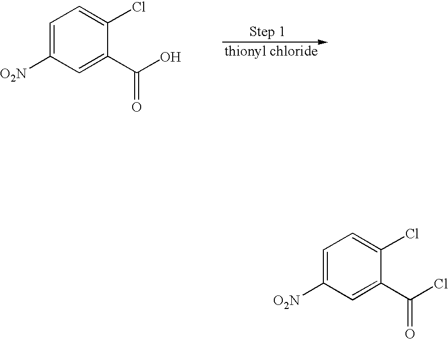

In a further step 2-chloro-5-nitrobenzoyl halide is coupled with the acetonide (2,2,6-trimethyl-1,3-dioxin-4-one) to produce a ketone, using a base such as lithium diisopropylamide, lithium hexamethyl disilylazine, or the like and in an acceptable organic solvent such as THF, to produce 6-[2-(2-chloro-5-nitrophenyl)-2-oxoethyl]-2,2-dimethyl-1,3-dioxine-4-one) (1), as follows:

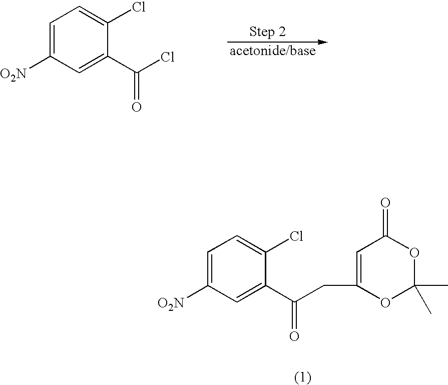

In another two steps the 1,3-dioxine ring is opened at the 1 position and condensed with the halogen atom on the neighboring ring by heating to about 80° C. in tert-butyl alcohol under nitrogen atmosphere to obtain t-butyl (6-nitro-4-oxo-2-chromen-2-yl)acetate (2) as follows:

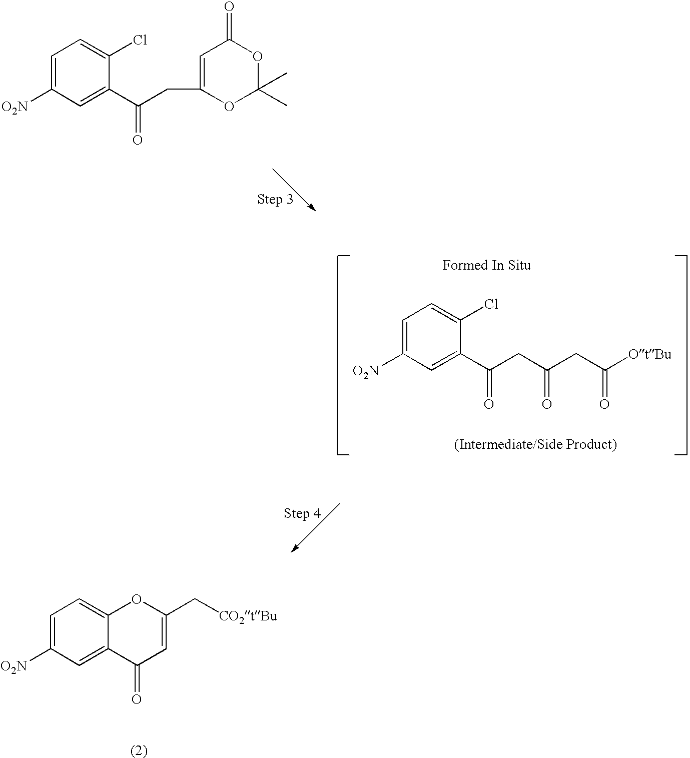

In another step the oxo, nitro and 2-3 alkene bond of the chromenone ring are reduced on the chromane t-butyl 2-(6-nitro-4-oxo-2-chromen-2-yl)acetate and the resulting 6-amino group is converted to an acetamido group in a single hydrogenation step. For example, a hydrogenation catalyst (such as 10% palladium on carbon) in the presence of glacial acetic acid followed by trifluoroacetic acid provides 2-(6-acetamido-chroman-2-yl) acetic acid (3) as follows:

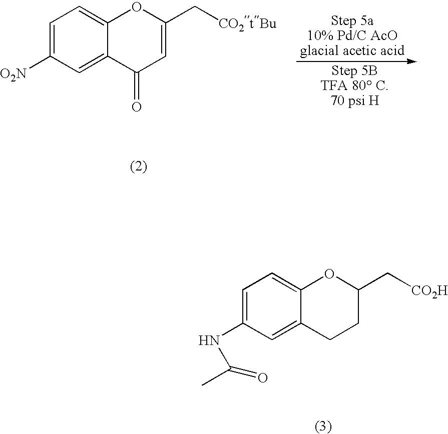

The amino protecting group can be removed with TFA, or the like, essentially as described in the paragraph bridging columns 147 and 148 of U.S. Pat. No. 5,731,324 followed by extraction with an organic solvent such as ethyl acetate, drying and concentrating the product to result in a dark oil of ethyl 2-(6-amino-chroman-2-yl)acetate, as follows:

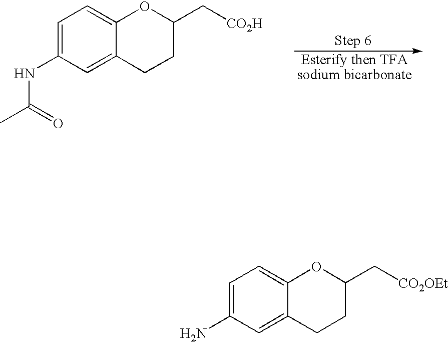

Optionally the amino group can be protonated to isolate the product as an amine acid halide salt or the like. The resulting ester may be converted to its corresponding acid or to another ester by methods known to those skilled in the art.

Enantiomeric Resolution and Acid Salt Formation

As is clear from the above formulae and the discussion above, the two-position acid ester group is attached to a chiral carbon which may optionally be resolved to produce a racemic mixture enriched in either the R or S enantiomers or completely resolved into a substantially pure composition of one of the enantiomers. Conventional processes may be utilized to resolve the enantiomers.

Compositions and Formulations

The compounds of this invention may be isolated as the free acid or base or converted to salts of various inorganic and organic acids and bases. Such salts are within the scope of this invention. Non-toxic and physiologically compatible salts are particularly useful although other less desirable salts may have use in the processes of isolation and purification.

A number of methods are useful for the preparation of the salts described above and are known to those skilled in the art. For example, reaction of the free acid or free base form of a compound of the structures recited above with one or more molar equivalents of the desired acid or base in a solvent or solvent mixture in which the salt is insoluble, or in a solvent like water after which the solvent is removed by evaporation, distillation or freeze drying. Alternatively, the free acid or base form of the product may be passed over an ion exchange resin to form the desired salt or one salt form of the product may be converted to another using the same general process.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

### Production of 2-chloro-5-nitrobenzoyl chloride

To a 3 L 3 neck round bottom flask equipped with a mechanical stirrer, nitrogen inlet, reflux condenser, heating mantle, vacuum system, and scrubber system for efficient removal of HCl and SO₂ gas which is liberated during the reaction, was charged, under nitrogen, 1.0 L (1.63 Kg, 13.7 moles) of thionyl chloride, and 1.3 Kg (6.4 moles) of 2-chloro-5-nitrobenzoic acid. The stirred mixture was placed under a N₂ flow, which was vented to the scrubber system. The stirred mixture was heated to reflux for 12 hours during which time the reaction slowly becomes complete. The clear yellow solution was placed under vacuum and excess thionyl chloride was removed by evaporation under vacuum. The resulting slurry was dissolved in 800 mL of toluene, and concentrated to dryness by evaporation under vacuum. The resulting yellow solid was dried at about 50° C. and about 20 mm Hg for about 4 hours to give 1.35 Kg of 2-chloro-5-nitrobenzoyl chloride as a yellow solid (95% yield).

### Example 2

### Production of 6-[2-(2-chloro-5-nitrophenyl)-2-oxoethyl]-2,2-dimethyl-1,3-dioxine-4-one) (1))

To a dried 1 liter 3 neck round bottom flask equipped with a mechanical stirrer, nitrogen inlet, 500 mL addition funnel, cooling system, thermowell, was charged under nitrogen, 300 mL of anhydrous THF, and 38.3 g (0.27 moles) of 2,2,6-trimethyl-1,3-dioxin-4-one (acetonide). The stirred reaction was cooled to about −60° C. and 240 mL of 1.5 M lithium diisopropylamide was added through the addition funnel. The addition funnel was rinsed with 25 mL of anhydrous tetrahydrofuran. The orange solution was stirred for 1.0 hours at −70° C. The addition funnel was charged with 40 g of 2-chloro-5-nitrobenzoyl chloride (acid chloride) dissolved in 50 mL of anhydrous THF. The acid chloride/THF solution was added to the reaction at a rate which maintains the reaction temperature<−60° C. (addition time=about 1 hour). The dark orange reaction solution was stirred for 0.5 hours and then cooling was removed. When the reaction temperature reaches −50° C., it was quenched with 80 mL of 6 M HCl. The reaction color becomes yellow-orange. The organic layer is separated and the aqueous layer is extracted one time with 100 mL of ethyl acetate. The organic layers were combined and washed with 100 mL of 10% NaHCO₃ followed by two washes with 100 mL of saturated aqueous sodium chloride solution. The organic layer was dried over 50 g of Na₂SO₄, filtered, and concentrated (about 25 mm Hg, <50° C.) to give 83 g of an orange oil. The oil was diluted with 40 mL of t-butyl methyl ether. The product started to crystallize immediately. After stirring at about 15° C. for about 2 hours, the slurry was filtered and the product was air dried to give 44.4 g of 6-[2-(2-chloro-5-nitrophenyl)-2-oxoethyl]-2,2-dimethyl-1,3-dioxine-4-one) (1) (75% yield), a yellow solid.

### Example 3

### Production of 6-[2-(2-chloro-5-nitrophenyl)-2-oxoethyl]-2,2-dimethyl-1,3-dioxine-4-one) (1))

To a dried 3 liter 3 neck round bottom flask equipped with an overhead stirrer, nitrogen bubbler, condenser, a 1 L addition funnel, cooling system, thermowell, was charged under nitrogen, 600 mL of anhydrous THF, and 40.2 g (0.250 moles, 88.2% pure) of 2,2,6-trimethyl-1,3-dioxin-4-one (acetonide). The dark solution was stirred and cooled to about −70° C. via acetone-dry ice bath and then 358 mL of 1.3 M lithium hexamethyidisilylazine (LiHMDS) (0.466 moles) was added over a 30 minute period. The LiHMDS was added at a rate which kept the temperature below −50° C. The dark solution was stirred for 1 hour while cooling to −65° C. and then 50.0 g of 2-chloro-5-nitrobenzoyl chloride (0.227 moles, acid chloride) dissolved in 60 mL of anhydrous THF was added to the reaction at a rate which maintains the reaction temperature<−50° C. (addition time=about 30 minutes). The dark orange reaction solution was stirred for 30 minutes hours and then was warmed to 35° C. to the dark solution was charged 42 mL of glacial acetic acid (0.73 moles). A slurry immediately formed and the exotherm raised the temperature to about 0° C. From the slurry was removed 400 mL of THF via vacuum distillation, keeping the temperature of the distillation at or below 25° C. The remaining sludge was diluted with 700 mL of dichloromethane. The slurry was transferred to a 22 L separatory funnel. The slurry was then washed with 500 mL of 1 M HCl/water (200 mL 2M HCl and 300 mL water). The organic layer was then washed with 1 L of brine/saturated NaHCO₃ (700 mL brine/300 mL sat'd NaHCO₃). The organic layer was then washed with 1 L of brine/1 M HCl (700 mL brine/300 mL 1M HCl). The organic layer was then dried with 50 g of Na₂SO₄ for two hours. The Na₂SO₄ was removed via filtration and the organic layer concentrated to a brown solid via vacuum distillation, keeping the temperature below 30° C. during concentration. The crude weight of the solid was 7.3 g (102% yield with solvent). The brown solid was washed with 200 mL of t-butyl methyl ether/hexane (1:1) and filtered. The resultant solid was dried at 50° C. under vacuum for 6 hours. The result was 70.8 g of a slightly brown solid of 6-[2-(2-chloro-5-nitrophenyl)-2-oxoethyl]-2,2-dimethyl-1,3-dioxine-4-one) (1) (95% yield).

### Example 4

### Larger Scale Production of 6-[2-(2-chloro-5-nitrophenyl)-2-oxoethyl]-2,2-dimethyl-1,3-dioxine-4-one) (1))

A 22 L 3 neck round bottom flask with similar equipment to that of Example 3, and 700 g (3.18 moles) of 2-chloro-5-nitrobenzoyl chloride starting material was used essentially as described in Example 3, to produce 770 g (2.32 moles) of 6-[2-(2-chloro-5-nitrophenyl)-2-oxoethyl]-2,2-dimethyl-1,3-dioxine-4-one) (73% isolated yield).

### Example 5

### Production of t-butyl 2-(6-nitro-4-oxo-2-chromen-2-yl)acetate (2)

To a 1 L 3 neck round bottom flask equipped with an overhead stirrer, nitrogen bubbler, condenser, and thermowell, was charged 250 mL of tert butyl alcohol. The stirring was started and then 50 g of 6-[2-(2-chloro-5-nitrophenyl)-2-oxoethyl]-2,2-dimethyl-1,3-dioxine-4-one) (0.15 moles), 30 g of dried 3A molecular sieves (powdered), and 50 g of dried Amberlyst A21 was charged. The slurry was heated to 80° C. and the reaction followed by TLC (70/30 hexane/ethyl acetate). Once the reaction was deemed complete by TLC, the heat was turned off. Once the reaction reached 50° C., it was filtered to remove the resin and sieves (NOTE—filtration was slow and pressure filtration may optionally be used). After filtration, the resin and sieves were washed three times with 1 L of ethyl acetate. The washes and filtrate were combined and concentrated under vacuum to provide 43 g of a brown solid (91% yield). The brown solid was washed with 100 mL of isopropanol/hexane (1:1) and filtered. The resultant beige solid was dried under maximum achievable vacuum at 50° C. for 6 hours. The result was 40 g of a beige solid of t-butyl 2-(6-nitro4-oxo-2-chromen-2-yl)acetate (2) (85% yield).

### Example 6

### Larger Scale Production of t-butyl 2-(6nitro-4-oxo-2-chromen-2-yl)acetate (2)

A 22 L 3 neck round bottom flask with similar equipment to that of Example 5, and 3.07 Kg (9.2 moles) of 6-[2-(2-chloro-5-nitrophenyl)-2-oxoethyl]-2,2-dimethyl-1,3-dioxine-4-one starting material was used essentially as described in Example 5, to produce 2.1 Kg (6.9 moles) of t-butyl 2-(6-nitro-4-oxo-2-chromen-2-yl)acetate (2) (75% isolated yield).

### Example 7

### Production of 2-(6-acetamido-chroman-2-yl) acetic acid (3)

To a 8 L parr hydrogenator was charged 600 g of t-butyl 2-(6-nitro-4-oxo-2-chromen-2-yl)acetate (2) (1.97 moles), 350 mL of acetic anhydride (3.77 moles), 90 g of 10% palladium on carbon, 400 g of dried 3 angstrom molecular sieves (powdered), and 3 L of glacial acetic acid. The bomb was sealed and then purged three times with nitrogen followed by three hydrogen purges. The reaction mixture was pressurized to 70 psi hydrogen and heated to 80° C. while stirring. The reaction was monitored by TLC and once all the double bond had been hydrogenated (typically 12 hours) the bomb was cooled to 50° C. The hydrogen was evacuated and the bomb purged three times with nitrogen.

The material remaining in the bomb and the bomb was charged with 350 mL of trifluoroacetic acid (4.54 moles). The bomb was re-sealed, purged three times with hydrogen and the pressurized to 70 psi hydrogen. The reaction mixture was heated to 80° C. while stirring. The reaction was monitored by HPLC. Once all of the alcohol was hydrogenated and ester hydrolyzed, the reaction temperature was cooled to room temperature. The hydrogen was evacuated and the bomb purged three times with nitrogen. The bomb was emptied and the reaction mixture was filtered through a celite bed. The catalyst and sieves were washed one time with 1 L of glacial acetic acid. The filtrate and wash were combined and concentrated down (about 25 mm Hg and about 75° C.) to a brownish-yellow oil. The oil was dissolved in 4 L of ethyl acetate and then the product was extracted out with 4 L of saturated NaHCO₃. The aqueous layer was washed one time with 2 L of ethyl acetate and then neutralized to a pH of 3-4 with concentrated HCl and then extracted three times with 3 L of ethyl acetate. The ethyl acetate extracts were combined and concentrated (at about 25 mm Hg and about 50° C.) to give a brown solid. The brown solid was washed with 600 mL of acetonitrile and then filtered to provide a white solid. The result after drying of the white solid at about 25 mm Hg and about 50° C. for 8 hours was 333 g of 2-(6-acetamido-chroman-2-yl) acetic acid (3) (68% yield).

### Example 8

### Production of ethyl 2-(6aminochroman-2-yl)acetate (4)

257 g of 2-(6-acetamido-chroman-2-yl) acetic acid as set forth in Example 7 is refluxed for 16 h under nitrogen in 2.7 L of sulfuric acid 3 N solution in absolute ethyl alcohol. The reaction mixture is then concentrated under reduced pressure (rotatory evaporator) to a whole mass of 1.03 Kg. Then 5 L of toluene and 430 g of sodium hydrogen carbonate in 1 L of water are added successively (at neutralization the reaction mixture becomes pink) After 10 minutes under stirring, the toluene is separated from the aqueous layer. The aqueous layer is then extracted successively 4 times with 1 L of toluene until completion of extraction (monitoring by HPLC to follow extraction process). The pooled toluenic phase is dried on magnesium sulfate and after filtration concentrated to 4 L. 430 mL of a 3.6 N hydrochloric acid ethereal solution is then added to precipitate the crude ethyl (6-amino-chroman-2-yl) acetate hydrochloride salt. After 1 hour of stirring at 20° C. the hydrochloride salt is filtrated and rinsed with 500 mL of toluene.

The rinsed hydrochloride salt is slurried in 2 L of aqueous sodium bicarbonate solution to neutralize the hydrochloride and release the hydrochloride amine salt as the free amine. The aqueous solution is extracted twice with 1 L of ethyl acetate. The organic layers are pooled, dried over sodium sulfate and concentrated under reduced pressure to leave 163.5 g (yield 88.3%) of a dark oil of racemic ethyl 2-(6-amino-chroman-2-yl)acetate having essentially the characteristics described in the paragraph bridging columns 147 and 148 of U.S. Pat. No. 5,731,324.

¹H-NMR (400 MHz, CDCl₃) 6.61 (d, J=8.9 Hz, 1H), 6.46 (dd, J=8.9 Hz, 2.5 Hz, 1H), 6.40 (d, J=2.5 Hz, 1H), 4.37 (qd, J=7.5 Hz, 1.2 Hz, 1H), 4.18 (q, J=7.2 Hz, 2H), 3.22 (s, 2H), 2.81 (ddd, J=16.5 Hz, 5.2 Hz, 4.1 Hz, 1H), 2.58 (dd, J=15.4 Hz, 7.4 Hz, 1H), 2.02 (dm, J=13.5 Hz, 1H), 1.75 (m, 1H), 1.07 (t, J=7.2 Hz, 3H). ¹³C-NMR (100 MHz, CDCl₃) 107.9, 147.5, 139.4, 122.1, 117.3, 115.9, 115.0, 72.1, 60.6, 40.6, 27.3, 24.5, 14.2.

In view of the above description it is believed that one of ordinary skill can practice the invention. The examples given above are non-limiting in that one of ordinary skill in view of the above will readily envision other permutations and variations on the invention without departing from the principal concepts. Such permutations and variations are also within the scope of the present invention.

1. A process for making a compound according to the formula 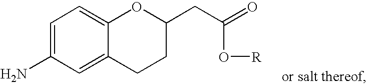
wherein R is H or an alkyl group, comprising:
(a) reacting 2-chloro-5-nitrobenzoic acid with a compound capable of halogenating the acid to form 2-chloro-5-nitrobenzoic acid halide as follows: 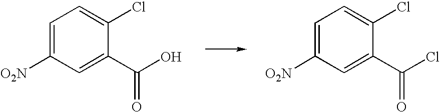
wherein the halogenating agent is a member selected from the group consisting of a metallic acid halide, thionyl halide, and an organic halide donor compound;
(b) coupling 2-chloro-5-nitrobenzoyl chloride of the product from (a) above with 2,2,6-trimethyl-1,3-dioxin-4-one to produce a ketone, in a lithium salt base in an acceptable organic solvent to produce 6-[2-(2-chloro-5-nitrophenyl)-2-oxoethyl]-2,2-dimethyl-1,3-dioxine-4-one, as follows: 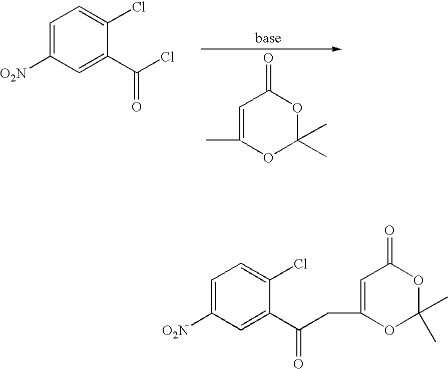
(c) opening the 1,3-dioxine ring of the product from (b) above and condensing the opened 1,3-dioxane ring with the halogen atom to effect ring closure by heating the product from (b) above to about 80° C. in tert butyl alcohol under nitrogen atmosphere to obtain t-butyl (6-nitro-4-oxo-2-chromen-2-yl)acetate (2), as follows: 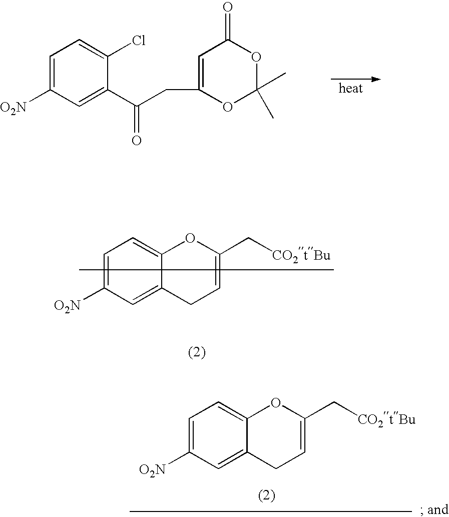
(d) reducing the 4-oxo group, 6-nitro group and 2-3 alkene bond of the chromenone ring of the product from (c) above in a single step or in separate steps as follows:

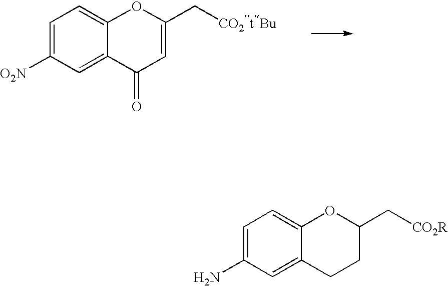

2. A process according to claim 1, wherein (d) comprises:
(d1) reducing at least the 6-nitro group in the presence of glacial acetic acid followed by hydrogenation using 10% palladium on carbon in the presence of trifluoro acetic acid to form the 6-acetamido group as follows: 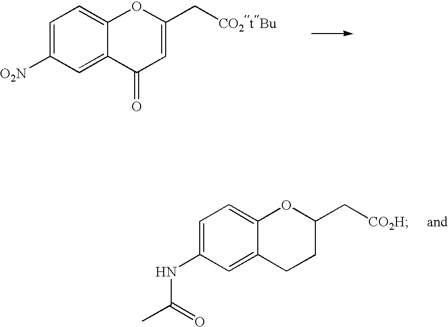
(d2) removing the protecting group from the 6-amino group by acidification and forming the final product as the free acid or ester as follows:

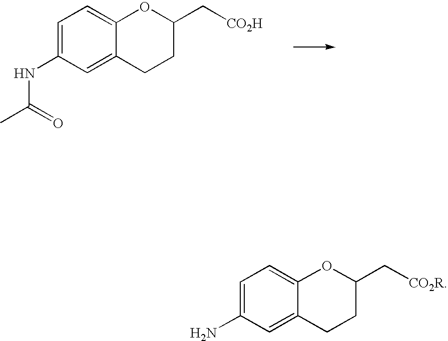

3. The process according to claim 2, wherein in (d2) the acidification is performed with trifluoroacetic acid at a temperature of about 40-80° C.

4. The process according to claim 2, comprising the additional step of forming a halide salt of the 6-amino group of the final product and isolating the salt as a polymorphic or crystalline material.

5. The process according to claim 2, wherein the lithium salt base in (b) is lithium diisopropylamide or lithium hexamethyl disilylazine, and the solvent is THF.

6. The process according to claim 1, wherein the lithium salt base in (b) is lithium diisopropylamide or lithium hexamethyl disilylazine, and the solvent is THF.

7. The process according to claim 1, comprising the additional step of forming a halide salt of the 6-amino group of the product from (d) and isolating the salt as a polymorphic or crystalline material.

8. The process according to claim 1, for making a compound according to the formula:

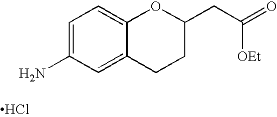

9. The process according to claim 2, wherein the halogenating agent of (a) is thionyl chloride, the base of (b) is lithium diisopropylamide or lithium hexamethyldisilylazine, the reaction solvent of (c) is t-butyl alcohol and the reaction temperature is about 80° C., and (d) is conducted in a two-step, single container hydrogenation process which produces the acetamido side chain and a free acid side chain.
